(19) United States
(12) Patent Application Publication
(10) Pub. No.: US 2022/0211647 A1
(43) Pub. Date: Jul. 7, 2022

(54) COMPOSITIONS AND METHODS FOR POTENTIATING DERIVATIVES OF 4-AMINOPHENOLS

(71) Applicant: SCISPARC LTD., Tel-Aviv (IL)

(72) Inventors: Ascher SHMULEWITZ, Tel Aviv (IL); Adi ZULOFF-SHANI, Ramat HaSharon (IL); Ephraim BRENER, Rishon LeZion (IL)

(73) Assignee: SCISPARC LTD., Tel-Aviv (IL)

(21) Appl. No.: 17/604,801

(22) PCT Filed: Apr. 22, 2020
(86) PCT No.: PCT/IB2020/000317

§ 371 Date: Oct. 19, 2021

Related U.S. Application Data
(60) Provisional application No. 62/837,274, filed on Apr. 23, 2019.

(51) Int. Cl. A61K 31/164; A61K 31/167; A61K 45/06; A61P 29/00

(52) U.S. Cl. CPC A61K 31/164; A61P 29/00; A61K 45/06; A61K 31/167

(57) ABSTRACT

The present disclosure provides pharmaceutical compositions and methods for treating paracetamol amenable conditions, such as pain and fever. The pharmaceutical compositions comprise certain combinations of derivatives of 4-Aminophenols and N-acylethanolamines. Also disclosed are methods and kits for treating or minimizing paracetamol amenable conditions.

### CROSS REFERENCE TO RELATED APPLICATIONS

This application is based on and claims benefit of priority of U.S. Provisional Patent Application No. 62/837,274, filed on Apr. 23, 2019, which is incorporated herein by reference in its entirety.

### FIELD OF THE INVENTION

The present disclosure relates to certain combinations of derivatives of 4-Aminophenols and N-acylethanolamines, and to their use in treating diseases, disorders, and conditions related but not limited to pain and fever, or otherwise amenable by paracetamol treatment. In particular, the present disclosure relates to pharmaceutical compositions containing fixed doses of paracetamol and palmitoylethanolamide and methods of treating relevant disorders with the disclosed compositions.

### BACKGROUND OF THE INVENTION

Paracetamol (acetaminophen) is a commonly used analgesic and antipyretic drug available without a prescription. Paracetamol and acetaminophen are the official names derived from its chemical name: N-acetyl-para-aminophenol or N-acetyl-para-aminophenol (Jóźwiak-Bebenista, 2014). Paracetomol is synthesized by reacting 4-Aminophenol with ethanoic anhydride.

Paracetamol was introduced into the pharmacological market as a prescribed analgesic and antipyretic drug for children under its trade name Tylenol Children's Elixir. On the World Health Organization (WHO) analgesic ladder, which precisely defines the rules for application of analgesic drugs, paracetamol has been placed on all three steps of pain treatment intensity (Jóźwiak-Bebenista, 2014). In varying pains of moderate intensity, paracetamol as a weak analgesic together with nonsteroidal analgesic drugs or coanalgesics (e.g., caffeine) is a basic non-opioid analgesic (the first step of the analgesic ladder). When pain maintains or increases, paracetamol is used as an additional analgesic with weak (e.g., caffeine, tramadol) or strong (e.g., morphine, phentanyl) opioids from the second and third step of the analgesic ladder, respectively. Moreover, paracetamol is the drug of choice in patients in whom application of nonsteroidal anti-inflammatory drugs (NSAIDs) are contraindicated, e.g., in the case of gastric ulcers, hypersensitivity to aspirin, impairments in blood coagulation, in pregnant women, nursing mothers, and children with fever accompanying a disease (Leung, 2012).

Due to the widespread use of paracetamol, and increasing dosage regimen of the drug, paracetamol related adverse events are reported more than ever. Acute overdoses of paracetamol can cause potentially fatal liver damage. In 2011, the U.S. Food and Drug Administration launched a public education program to help consumers avoid overdose, warning: “Acetaminophen can cause serious liver damage if more than directed is used.” The FDA immediately required manufacturers to update labels of all prescription combination acetaminophen products to warn of the potential risk for severe liver injury and required that such combinations contain no more than 325 mg of acetaminophen. In addition, paracetamol use and asthma has been linked, but whether this association is causal is still debated at this time (Sheehan, 2016). In 2013, the FDA issued a new warning about paracetamol, stating that the drug could cause rare and possibly fatal skin reactions such as Stevens-Johnson syndrome and toxic epidermal necrolysis.

Although paracetamol was discovered over 100 years ago and has been widely used in medical practice for more than half the century, its mechanism of action is only now being elucidated. While paracetamol has analgesic and antipyretic properties similar to NSAIDs, it does not inhibit the function of any cyclooxygenase (COX) enzyme outside of the central nervous system, and thus does not possess anti-inflammatory activity like NSAIDs (Ghanem, 2016).

Studies on the mechanism of action of paracetamol suggest that it acts as a pro-drug because its active metabolites demonstrate an association with the endocannabinoid system. Paracetamol has been shown to modulate the endogenous cannabinoid system in the brain through paracetamol's metabolite, arachidonoylphenolamine (AM404). AM404 inhibits the reuptake of the cannabinoid (anandamide) by neurons, making it more available to reduce pain (Ghanem, 2016). Furthermore, different concentrations of AM404 have been found to inhibit COX-1 and COX-2 enzymes. In these areas of the brain an increased production of the active metabolite AM404 can be found, and this in turn may to a certain degree explain the inhibitory action of paracetamol towards cyclooxygenases in the CNS (Bertolini, 2006).

The inventors have found that derivatives of 4-Aminophenols, including paracetamol, unexpectedly collaborate with other molecules of the endocannabinoid system in potentiating its analgesic efficacy. This collaboration reminds the known in the field phenomenon called “entourage effect”. The basic idea of the “entourage effect” is that cannabinoids within the cannabis plant work together, or possess synergy, and affect the body in a mechanism similar to the body's own endocannabinoid system (Ben-Shabata, 1998). The entourage effect within the endocannabinoid system provides a number of benefits, including the ability to affect multiple targets within the body, to improve the absorption of active ingredients, and to minimize adverse side effects of one of the components/compounds.

N-acylethanolamines (NAEs) are lipid-derived signaling molecules. They are formed when one of several types of acyl groups are linked to the nitrogen atom of ethanolamine (Okamoto, 2004). NAEs are generated by the membrane enzyme NAPE-PLD, and natural bile acids regulate this process (Magotti, 2014).

Palmitoylethanolamide (PEA, also known as N-(2-hydroxyethyl)hexadecanamide; Hydroxyethylpalmitamide; Palmidrol; N-palmitoylethanolamine; and Palmitylethanolamide) is an endogenous fatty acid amide, belonging to the class of nuclear factor agonists. PEA has been demonstrated to bind to a receptor in the cell-nucleus (a nuclear receptor) and exerts a variety of biological functions related to chronic pain and inflammation. Studies have shown that PEA interacts with distinct non-CB1/CB2 receptors Studies have also shown that PEA production and inactivation can occur independently of AEA and 2-AG production and inactivation. Much of the biological effects of PEA on cells can be attributed to its affinity to PPAR (O'Sullivan, 2007). PEA has affinity to cannabinoid-like G-coupled receptors GPR55 and GPR119 as well as the transient receptor potential vanilloid type 1 receptor (TRPV1) (Godlewski, 2009). PEA exhibits anti-inflammatory, anti-nociceptive, neuro-protective, and anti-convulsant properties.

There remains a need in the field of pain and fever management for combination treatments of 4-Aminophenols, such as paracetamol, and other agents capable of lowering the dosage regimen of the 4-Aminophenol and reducing adverse events, while maintaining or improving the therapeutic efficacy of paracetamol.

The current disclosure provides a combined therapy of paracetamol and PEA to either improve paracetamol stand-alone induced analgesia and fever relief, prolong the therapeutic window of either agent, or reduce the required dose of paracetamol to achieve the desired effects.

### SUMMARY OF THE INVENTION

The present disclosure provides pharmaceutical compositions and dosage forms comprising derivatives of 4-Aminophenols and N-acylethanolamines for use in treating diseases, disorders, and conditions related but not limited to pain and fever, or otherwise amenable by paracetamol treatment.

The present disclosure is based in part on experimental findings that certain combinations of derivatives of 4-Aminophenols and N-acylethanolamines enhance the biological activity of the derivatives of 4-Aminophenols as an analgesic drug and/or reduce its associated side effects.

The present disclosure provides, in one aspect, a pharmaceutical composition comprising a therapeutically-effective amount of a mixture of at least one derivative of 4-Aminophenol or a salt thereof and at least one N-acylethanolamine or a salt thereof.

In certain embodiments, the pharmaceutical composition comprises about 0.5 mg to about 4000 mg of at least one derivative of 4-Aminophenol or a salt thereof. In certain embodiments, the at least one derivative of 4-Aminophenol is paracetamol. In certain embodiments, the pharmaceutical composition comprises about 10 mg, 48 mg, 80 mg, 120 mg, 160 mg, 325 mg, 500 mg, or 650 mg paracetamol. Each possibility represents a separate embodiment of the disclosure.

In certain embodiments, the pharmaceutical composition comprises about 800 mg of the N-acylethanolamine or salt thereof. In certain embodiments, the at least one N-acylethanolamine is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, and any combination thereof. Each possibility represents a separate embodiment of the disclosure. In certain embodiments, the at least one N-acylethanolamine is PEA or a salt thereof.

In certain embodiments, the pharmaceutical composition comprises a mixture of paracetamol or a salt thereof and PEA or a salt thereof. In certain embodiments, the mixture comprises about 0.5 mg to about 4000 mg paracetamol or a salt thereof and about 50 mg to about 5000 mg PEA or a salt thereof.

In certain embodiments, the pharmaceutical composition is formulated for systemic administration. In certain embodiments, the pharmaceutical composition is formulated for oral, vaginal, rectal, oral mucosal, nasal, sublingual, inhalational, topical, parenteral, intravenous, intramuscular, or subcutaneous administration. Each possibility represents a separate embodiment of the disclosure. In certain embodiments, the pharmaceutical composition is formulated for oral, vaginal, or rectal administration. In certain embodiments, the pharmaceutical composition is formulated as a solution or as a suppository.

Embodiments of the present disclosure further provide a dosage unit comprising the pharmaceutical composition described above.

The present disclosure further provides, in another aspect, a pharmaceutical composition as described above for use in a method for treating a pain. In certain embodiments, the pain is an acute pain, chronic pain, or neuropathic pain. In certain embodiments, the pharmaceutical composition is used in treating at least side-effect associated with paracetamol consumption. In certain embodiments, the pharmaceutical composition is used in a method for treating a fever. In certain embodiments, the pharmaceutical composition is used in the manufacture of a medicament for treating pain or fever in a subject in need of such treatment.

The present disclosure further provides, in another aspect, a method of treating pain or fever comprising administering to a subject in need thereof a therapeutically-effective amount of at least one derivative of 4-Aminophenol or salt thereof and administering to the subject a therapeutically-effective amount of at least one N-acylethanolamine or a salt thereof.

In certain embodiments, the therapeutically-effective amount of the derivative of 4-Aminophenol or a salt thereof is from about 0.5 mg to about 4000 mg. In certain embodiments, the at least one derivative of 4-Aminophenol is paracetamol. In certain embodiments, the therapeutically-effective amount of paracetamol is about 10 mg, 48 mg, 80 mg, 120 mg, 160 mg, 325 mg, 500 mg, or 650 mg. Each possibility represents a separate embodiment of the disclosure.

In certain embodiments, the pharmaceutical composition comprises about 800 mg of the N-acylethanolamine or salt thereof. In certain embodiments, the at least one N-acylethanolamine is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, and any combination thereof. Each possibility represents a separate embodiment of the disclosure. In certain embodiments, the at least one N-acylethanolamine is PEA or a salt thereof.

In certain embodiments, the method comprises administering a mixture of paracetamol or a salt thereof and PEA or a salt thereof. In certain embodiments, the mixture comprises about 0.5 mg to about 4000 mg paracetamol or a salt thereof and about 50 mg to about 5000 mg PEA or a salt thereof.

In certain embodiments, the derivative of 4-Aminophenol and N-acylethanolamine are formulated for systemic administration. In certain embodiments, the derivative of 4-Aminophenol and N-acylethanolamine are formulated for oral, vaginal, rectal, oral mucosal, nasal, sublingual, inhalational, topical, parenteral, intravenous, intramuscular, or subcutaneous administration. Each possibility represents a separate embodiment of the disclosure. In certain embodiments, the derivative of 4-Aminophenol and N-acylethanolamine are formulated for oral, vaginal, or rectal administration. In certain embodiments, the derivative of 4-Aminophenol and N-acylethanolamine are formulated as a solution or as a suppository.

In certain embodiments, the method described above is used for preventing or treating at least one side-effect associated with paracetamol consumption in a human subject in need thereof. In certain embodiments, side-effects associated with paracetamol consumption are liver and/or kidney damage. In certain embodiments, side-effect associated with paracetamol consumption are worsening of asthma in asthma patients. In certain embodiments, side-effects associated with paracetamol consumption are skin reactions such as Stevens-Johnson syndrome and toxic epidermal necrolysis. In certain embodiments, side-effects associated with paracetamol consumption are allergic reactions, which can cause rashes and swelling. In certain embodiments, side-effects associated with paracetamol consumption are flushing, low blood pressure, and a fast heartbeat, when the paracetamol is given intravenously.

In certain embodiments, the derivative of 4-Aminophenol and N-acylethanolamine as described in the method above are orally administered. In certain embodiments, the derivative of 4-Aminophenol and N-acylethanolamine are administered daily. In certain embodiments, the derivative of 4-Aminophenol and N-acylethanolamine are comprised in the same pharmaceutical composition.

The present disclosure further provides, in another aspect, a kit for the treatment of pain or fever comprising a pharmaceutical composition comprising a therapeutically-effective amount of at least one derivative of 4-Aminophenol or a salt thereof, a pharmaceutical composition comprising a therapeutically-effective amount of at least one N-acylethanolamine or a salt thereof, and instructions for administering the derivative of 4-Aminophenol and N-acylethanolamine.

Other objects, features, and advantages of the present disclosure will become clear from the following description.

### DETAILED DESCRIPTION OF THE INVENTION

The present disclosure provides pharmaceutical compositions and dosage forms, comprising at least one derivative of 4-Aminophenol and at least one N-acylethanolamine. The present disclosure further provides methods for the use of these compositions and dosage forms in treating diseases, disorders, and conditions related but not limited to pain and fever, or otherwise amenable by paracetamol treatment.

The pharmaceutical compositions of the disclosed embodiments provide an improved medicament compared to current therapies, exhibiting an increased therapeutic activity, while minimizing administered paracetamol dosages and reducing paracetamol-associated adverse events. The embodiments described herein are based on the discovery that N-acylethanolamine compounds exhibit a 4-Aminophenol (paracetamol)-sparing effect. The phrase “paracetamol-sparing effect” as used herein refers to the enablement of the use of low dosages of the named compounds in instances wherein a mid- or high-dosages of the compounds are typically required. The paracetamol and N-acylethanolamine compounds according to the present disclosure include pharmaceutically acceptable forms thereof, including isomers such as diastereomers and enantiomers, salts, solvates, and polymorphs, as well as racemic mixtures.

According to one aspect, the present disclosure provides a pharmaceutical composition comprising paracetamol, N-acylethanolamine, and an acceptable pharmaceutical carrier.

The present disclosure provides, in one aspect, a pharmaceutical composition comprising a therapeutically-effective amount of a mixture of at least one derivative of 4-Aminophenols or a salt thereof and at least one N-acylethanolamine or a salt thereof.

As used herein, a “pharmaceutical composition” refers to a preparation of the active agents described herein with other chemical components such as physiologically suitable carriers and excipients. The purpose of a pharmaceutical composition is to facilitate administration of a compound to an organism. As used herein, the phrase “pharmaceutically acceptable carrier” refers to a carrier, an excipient, or a diluent that does not cause significant irritation to an organism and does not abrogate the biological activity and properties of the administered compound. An adjuvant is included under these phrases.

The term “excipient” as used herein refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

The term “derivative” as used herein means a compound whose core structure is the same as, or closely resembles that of a reference compound, but which has a chemical or physical modification, such as different or additional side groups.

The term “carrier” as used herein refers to a diluent, adjuvant, excipient, or vehicle with which the compound is administered. Such pharmaceutical carriers can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The phrase “pharmaceutically acceptable” as used herein refers to molecular entities and compositions that are physiologically tolerable and do not typically produce an allergic or similar toxicity when administered to an individual. Preferably, and particularly where a formulation is used in humans, the term “pharmaceutically acceptable” may mean approved by a regulatory agency (for example, the U.S. Food and Drug Agency) or listed in a generally recognized pharmacopeia for use in animals (e.g., the U.S. Pharmacopeia).

The term “derivative of 4-Aminophenols” or “4-Aminophenols” as used herein generally refers to the organic compound with the formula H2NC6H40H. Prominently, it is the final intermediate in the industrial synthesis of paracetamol. Treating 4-Aminophenol with acetic anhydride gives paracetamol. 4-Aminophenol is one of three isomeric aminophenols, the other two being 2-Aminophenol and 3-Aminophenol.

The term “N-acylethanolamine” as used herein generally refers to a type of fatty acid amide, lipid-derived signaling molecules, formed when one of several types of acyl groups are linked to the nitrogen atom of ethanolamine. These amides conceptually can be formed from a fatty acid and ethanolamine with the release of a molecule of water, but the known biological synthesis uses a specific phospholipase D to cleave the phospholipid unit from N-acylphosphatidylethanolamines (NAPEs, hormones released by the small intestine into the bloodstream when it processes fat). The suffixes -amine and -amide in these names each refer to the single nitrogen atom of ethanolamine that links the compound together: it is termed “amine” in ethanolamine because it is considered a free terminal nitrogen in that subunit, while it is termed “amide” when it is considered in association with the adjacent carbonyl group of the acyl subunit. Names for these compounds may be encountered with either “amide” or “amine” in the present application. The term “ethanolamine” is used in the generic sense and is meant to include mono-ethanolamine, di-ethanolamine, tri-ethanolamine, and mixtures thereof.

The term “salt” as used herein refers to any form of an active ingredient in which the active ingredient assumes an ionic form and is coupled to a counter ion (a cation or anion) or is in solution. This also includes complexes of the active ingredient with other molecules and ions, in particular complexes which are complexed by ion interaction.

In certain embodiments, the pharmaceutical composition described above comprises about 800 mg N-acylethanolamine or a salt thereof. In certain embodiments, the N-acylethanolamine is selected from the group consisting of N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, and any combination thereof. Each possibility represents a separate embodiment of the disclosure. In certain embodiments, the N-acylethanolamine is PEA or a salt thereof.

In certain embodiments, the mixture comprises paracetamol or a salt thereof and PEA or a salt thereof. In certain embodiments, the mixture comprises about 0.5 mg to about 4000 mg of a derivative of 4-Aminophenol or a salt thereof and about 50 mg to about 5000 mg PEA or a salt thereof. In certain embodiments, the mixture comprises about 10 mg to about 650 mg of a derivative of 4-Aminophenol or a salt thereof and about 250-2000 mg PEA or a salt thereof. In certain embodiments, the disclosed pharmaceutical composition is formulated for systemic administration. In certain embodiments, the disclosed pharmaceutical composition is formulated for oral, vaginal, rectal, oral mucosal, nasal, sublingual, inhalational, topical, parenteral, intravenous, intramuscular, or subcutaneous administration. In certain embodiments, the disclosed pharmaceutical composition is formulated for oral, vaginal, or rectal administration. In certain embodiments, the disclosed pharmaceutical composition is formulated as a solution or as a suppository. Each possibility represents a separate embodiment of the disclosure.

The present disclosure further provides, in another aspect, a dosage unit comprising or consisting of any one of the pharmaceutical compositions described above. Techniques for formulation and administration of drugs are well known in the art, and may be found, e.g. in “Remington's Pharmaceutical Sciences,” Mack Publishing Co., Easton, Pa.

Pharmaceutical compositions of the present disclosure may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present disclosure may be formulated in a conventional manner using one or more physiologically acceptable carriers comprising excipients and auxiliaries, which facilitate processing of the active ingredients into preparations that can be used pharmaceutically. Proper formulation is dependent upon the route of administration chosen.

For topical application, the active ingredients of the pharmaceutical composition may be formulated in crémes, ointments, solutions, patches, sprays, lotions, liniments, varnishes, solid preparations such as silicone sheets, and the like.

The term “topical” as used herein refers to the application of a disclosed composition directly onto at least a portion/region of a subject's skin (human's or non-human's skin) so as to achieve a desired effect, for example, treating dermatological diseases as described herein.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The term “mucosal administration” relates to delivery of a composition to a mucous membrane, such as the buccal or labial mucosa or the mucosa of the respiratory tract, such as the nasal mucosa.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose (HPMC), and sodium carbomethylcellulose (CMC); and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

The term “oral administration” refers to any method of administration in which an active agent can be administered by swallowing, chewing, sucking, or drinking an oral dosage form. Examples of solid dosage forms include conventional tablets, multi-layer tablets, capsules, caplets, etc., which do not substantially release the drug in the mouth or in the oral cavity.

Dragee cores are provided with suitable coatings. For this purpose, concentrated sugar solutions may be used which may optionally contain gum arabic, talc, polyvinyl pyrrolidone, CARBOPOL gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include stiff or soft, sealed capsules made of gelatin and a plasticizer, such as glycerol or sorbitol. The capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal and sublingual administration, the compositions may take the form of tablets or lozenges formulated in a conventional manner or in adhesive carriers.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethylcellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The present compositions can also be delivered using an in situ formed depot (ISFD). Examples of in situ formed depots include semi-solid polymers which can be injected as a melt and form a depot upon cooling to body temperature. The requirements for such ISFD include low melting or glass transition temperatures in the range of 25-658° C. and an intrinsic viscosity in the range of 0.05-0.8 dl/g. Below the viscosity threshold of 0.05 dl/g no delayed diffusion could be observed, whereas above 0.8 dl/g the ISFD was no longer injectable using a needle. At temperatures above 378° C. but below 658° C. these polymers behave like viscous fluids which solidify to highly viscous depots. Drugs are incorporated into the molten polymer by mixing without the application of solvents. Thermoplastic pastes (TP) can be used to generate a subcutaneous drug reservoir from which diffusion occurs into the systemic circulation.

In situ cross-linked polymer systems utilize a cross-linked polymer network to control the diffusion of macromolecules over a prolonged period of time. Use of in situ cross-linking implants necessitates protection of the bioactive agents during the cross-linking reaction. This could be achieved by encapsulation into fast degrading gelatin microparticles.

An ISFD can also be based on polymer precipitation. A water-insoluble and biodegradable polymer is dissolved in a biocompatible organic solvent to which a drug is added forming a solution or suspension after mixing. When this formulation is injected into the body the water miscible organic solvent dissipates and water penetrates into the organic phase. This leads to phase separation and precipitation of the polymer forming a depot at the site of injection. One example of such a system is ATRIGELE.

Thermally induced gelling systems can also be used as ISFDs. Numerous polymers show abrupt changes in solubility as a function of environmental temperature. The prototype of a thermosensitive polymer is poly(N-isopropyl acryl amide), poly-NIPAAM, which exhibits a rather sharp lower critical solution temperature.

Thermoplastic pastes such as the new generation of poly(ortho esters) developed by AP Pharma can also be used for depot drug delivery. Such pastes include polymers that are semi-solid at room temperature, hence heating for drug incorporation and injection is no longer necessary. Injection is possible through needles no larger than 22 gauge. The drug can be mixed into the systems in a dry and, therefore, stabilized state. Shrinkage or swelling upon injection is thought to be marginal and, therefore, the initial drug burst is expected to be lower than in the other types of ISFD. An additional advantage is afforded by the self-catalyzed degradation by surface erosion.

The compositions of the present disclosure can also be delivered from medical devices, such as orthopedic implants, contact lenses, micro needle arrays, patches and the like.

Sustained-release (SR), extended-release (ER, XR, or XL), time-release or timed-release, controlled-release (CR), or continuous-release (CR) pills are tablets or capsules formulated to dissolve slowly and release a drug over time. Sustained-release tablets are formulated so that the active ingredient is embedded in a matrix of insoluble substance (e.g. acrylics, polysaccharides, etc.) such that the dissolving drug diffuses out through the holes in the matrix. In some SR formulations the matrix physically swells up to form a gel, so that the drug has first to dissolve in matrix, then exit through the outer surface. The difference between controlled release and sustained release is that controlled release is perfectly zero order release. That is, the drug releases with time irrespective of concentration. On the other hand, sustained release implies slow release of the drug over a time period. It may or may not be controlled release.

Pharmaceutical compositions suitable for use in the context of the present disclosure include compositions wherein the active ingredients are contained in an amount effective to achieve the intended purpose. More specifically, a “therapeutically effective amount” means an amount of active ingredients effective to prevent, alleviate, or ameliorate symptoms or side effects of a disease or disorder, or prolong the survival of the subject being treated. Determination of a therapeutically effective amount is well within the capability of those skilled in the art, especially in light of the detailed disclosure provided herein.

For any preparation used in the methods of the disclosure, the dosage or the therapeutically effective amount can be estimated initially from in vitro and cell culture assays. For example, a dose can be formulated in animal models to achieve a desired concentration or titer. Such information can be used to more accurately determine useful doses in humans.

The dosage of each compound of the claimed combinations depends on several factors, including: the administration method, the disease to be treated, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

The dosage of paracetamol within the claimed combination when taken intravenously may be ranged from 0.5 mg to 4000 mg paracetamol per 50 kg subject daily, within maximal single dose of 1000 mg or 75 mg/kg per 24 hours.

The dosage of paracetamol within the claimed combination when taken orally may be ranged from 0.5 mg to 4000 mg paracetamol per 50 kg subject daily, within maximal single dose of 1000 mg per 24 hours.

The dosage of paracetamol within the claimed combination when taken via rectal route may be ranged from 0.5 mg to 3900 mg paracetamol per 24 hours or 650 mg every 4 to 6 hours.

The dosage of N-acylethanolamine, for example, PEA within the claimed combination may be ranged from 200 mg to 5000 mg PEA per subject daily.

Continuous daily dosing may not be required; a therapeutic regimen may require cycles, during which time a drug is not administered, or therapy may be provided on an as-needed basis during periods of acute disease worsening.

The present disclosure further provides, in another aspect, a pharmaceutical composition described above, or a dosage unit described above, for use in a method for treating a paracetamol amenable condition, including but not limited to pain, fever, or osteoarthritis.

Neuropathic pain is a localized sensation of unpleasant discomfort caused by damage or disease that affects the somatosensory system. The International Association for the Study of Pain's (IASP) widely used definition of pain states: “Pain is an unpleasant sensory and emotional experience associated with actual or potential tissue damage, or described in terms of such damage.” Accordingly, the term “pain,” as used herein, means an unpleasant sensory and emotional experience associated with actual or potential tissue damage, or described in terms of such damage. Neuropathic pain may be associated with abnormal sensations called dysesthesia, and pain from normally non-painful stimuli (allodynia). It may have continuous and/or episodic (paroxysmal) components and resemble stabbings or electric shocks. Common qualities include burning or coldness, “pins and needles” sensations, numbness, and itching. Nociceptive pain, by contrast, is more commonly described as aching. Central neuropathic pain is found in spinal cord injury, multiple sclerosis, and some strokes. Aside from diabetes and other metabolic conditions, the common causes of painful peripheral neuropathies are herpes zoster infection, HIV-related neuropathies, nutritional deficiencies, toxins, remote manifestations of malignancies, immune mediated disorders and physical trauma to a nerve trunk. Neuropathic pain is common in cancer as a direct result of cancer on peripheral nerves (e.g., compression by a tumor), or as a side effect of chemotherapy (chemotherapy-induced peripheral neuropathy), radiation injury, or surgery.

Fever, also known as pyrexia and febrile response, is defined as having a temperature above the normal range due to an increase in the body's temperature set-point.

In certain embodiments, a fever is caused by medical conditions ranging from not serious to potentially serious. This includes viral, bacterial, and parasitic infections such as the common cold, urinary tract infections, meningitis, malaria, and appendicitis among others. Non-infectious causes include vasculitis, deep vein thrombosis, side effects of medication, and cancer among others.

The term “treating” as used herein, includes, but is not limited to, any one or more of the following: abrogating, ameliorating, inhibiting, attenuating, blocking, suppressing, reducing, delaying, halting, alleviating or preventing one or more symptoms or side effects of the diseases or conditions of the disclosed embodiments.

The present disclosure further provides, in another aspect, a pharmaceutical composition described above, or a dosage unit described above, for use in a method for treating pain, fever, or osteoarthritis.

In certain embodiments, the therapeutic potency of paracetamol in the pharmaceutical composition is increased compared to the therapeutic potency of the same paracetamol in a similar pharmaceutical composition without the N-acylethanolamine. In certain embodiments, the required therapeutic dosage of paracetamol in the pharmaceutical composition is decreased compared to the required therapeutic dosage of the same paracetamol in a similar pharmaceutical composition without the N-acylethanolamine. In certain embodiments, at least one side-effect of paracetamol in the pharmaceutical composition is reduced compared to the same side-effect of the same paracetamol in a similar pharmaceutical composition without the N-acylethanolamine. In certain embodiments, the therapeutic window of paracetamol in the pharmaceutical composition is expended compared to the therapeutic window of the same paracetamol in a similar pharmaceutical composition without the N-acylethanolamine.

The present disclosure further provides, in another aspect, a method for treating a pain and/or a fever-related condition in a human subject in need thereof, the method comprising the step of administering to the subject a therapeutically-effective amount of a combination of a pharmaceutical composition comprising paracetamol or a salt thereof and a pharmaceutical composition comprising at least one N-acylethanolamine or a salt thereof, thereby treating a pain and/or a fever related condition.

Dosage escalation may or may not be required; a therapeutic regimen may require reduction in medication dosage.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (Fingl, 1975)

Depending on the severity and responsiveness of the condition to be treated, dosing can be of a single or a plurality of administrations, with course of treatment lasting from several days to several weeks, or until cure is effected or diminution of the disease state is achieved.

Suitable routes of administration may, for example, include oral, rectal, vaginal, topical, nasal, trans-nasal, transmucosal, intestinal, or parenteral delivery, including intramuscular, subcutaneous, and intramedullary injections, as well as intrathecal, direct intraventricular, intravenous, intraperitoneal, intranasal, intraocular injections or by means of inhalation or aspiration (smoking). Alternately, the pharmaceutical composition may be administered locally, rather than in a systemic manner, for example, via injection of the pharmaceutical composition directly into a tissue region of a patient.

In certain embodiments, paracetamol and the N-acylethanolamine are orally administered. In certain embodiments, paracetamol and the N-acylethanolamine are intravenously administered. In certain embodiments, paracetamol and the N-acylethanolamine are administered via rectal route. In certain embodiments, paracetamol and the N-acylethanolamine are daily administered. In certain embodiments, paracetamol and the N-acylethanolamine are comprised in the same pharmaceutical composition.

Compositions of the present disclosure may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the disclosure formulated in a pharmaceutically acceptable carrier may also be prepared, placed in an appropriate container, and labeled for treatment of an indicated inflammatory disorder, as further detailed above.

According to additional aspect, the present disclosure provides a kit comprising:
(i) a pharmaceutical composition comprising a paracetamol and a pharmaceutical acceptable carrier, and
(ii) a pharmaceutical composition comprising an N-acylethanolamine and a pharmaceutical acceptable carrier.

In certain embodiments, the kit further comprises written instructions for its use in the treatment of one or more pain condition.

In certain embodiments, the kit further comprises written instructions for its use in the treatment of one or more fever condition.

The foregoing description of the specific embodiments will so fully reveal the general nature of the compositions and methods that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

The following examples are presented in order to more fully illustrate some embodiments of the disclosure. They should, in no way be construed, however, as limiting the broad scope of the disclosure. One skilled in the art can readily devise many variations and modifications of the principles disclosed herein without departing from the scope of the disclosure.

### Examples

### Example 1. Evaluation of the Analgesic Effect of the Paracetamol and N-Palmitoylethanolamine (PEA) Combination in an Inflammation Rat Model (Acute/Chronic Pain)

The objective of the study is to evaluate the potential of a paracetamol-sparing effect using PEA in an inflammatory rat model (injection of complete Freund's adjuvant (CFA) as a model of monoarthritis).
Study variables and end points: mortality and morbidity are measured once a day. Clinical observations are made daily, with special attention given for signs of limping, infection, or edema in the injected subject. Body weight measurements are performed throughout the study, specifically upon arrival, before study initiation, and once a week thereafter upon study termination. Pain response endpoints are tested using incapacitance (weight bearing test) and tactile allodynia (von Frey test) at Day 0 (baseline) and Days 1, 3, 6, and 10. Upon study termination (Day 10), gross pathology and necropsy will be performed, examining the local injection site and major tissue and organ systems.
The principle of the study is based on the knowledge that injection of CFA induces acute and chronic inflammatory pain, which is used as a model for monoarthritis. The CFA induced inflammation follows a biphasic course, starting with an acute inflammatory reaction within hours that subsides after 3 days, and a chronic reaction that can last two-weeks and up to several months (Neugebauer, 2007).
Animal Handling: Animal handling is performed according to guidelines of the National Institute of Health (NIH) and the Association for Assessment and Accreditation of Laboratory Animal Care (AAALAC), and Pharmaseed's SOPs. Animals are housed in individual ventilated cages (IVC) (maximum 3 rats/cage) measuring 42.5×26.5×18.5 cm, with stainless steel top grill facilitating pelleted food and drinking water in plastic bottles; bedding: steam sterilized clean paddy husk (Envigo, Teklad, Laboratory grade, Sani-chips). Bedding material is changed along with the cage at least twice a week. Animals are fed ad libitum a commercial rodent diet (Teklad Certified Global 18% Protein Diet, Harlan cat #2018SC). Animals have free access to sterilized and acidified drinking water (pH between 2.5 and 3.5) obtained from the municipality supply and treated according to Pharmaseed's SOP No. 214: ‘Water system.”
Study Design: Rats are randomly allocated to cages on the day of reception. Allocation to relevant groups is done on Day 0, according to pain response testing results. Animals are allocated to seven treatment groups as indicated below:

1M Control (naïve)

2M Contol (induced)

3M Paracetamol (regular dose (RD))

4M Paracetamol (sub-effective (SED))

5M Paracetamol (high dose (overdose) (OD))

6M PEA

7M Paracetamol RD with PEA

8M Paracetamol SED with PEA

9M Paracetamol OD with PEA

CFA-induced inflammatory monoarthritis model causes moderate pain. As this model measures the ability of compounds to reduce pain, Buprenorphine (1 mg/kg) will be administered twice on the day of procedures only. Buprenorphine was chosen due to its short term pain relief effect, in addition to the fact that it has no anti-inflammatory effect, unlike other analgesic agents, such as meloxicam.
Anesthesia is induced for each animal by a chamber induction technique using inhalation anesthesia (Isoflurane at 5.0%). During surgery, the animal is maintained with Isoflurane at a level between 1.5 and 3.5%, with an oxygen flow rate of 1-2 liters/minute. 50 μL of Complete Freund's Adjuvant (CFA) is injected intra-articulary (IA) into the tibio-tarsal joint using a 27 gauge needle, according to the method described by Butler et al. (1992).
Morbidity and mortality checks are performed once a day. The animals, which will be humanely killed during the test, are considered for the interpretation of test results as animals that died during the test. In case of mortality before study scheduled termination, gross pathology evaluation is performed as close as possible to the time of death. The time of death will be recorded as precisely as possible.
The animals are observed for toxic/adverse symptoms daily, until study termination. Special attention is required for signs of limping, infection or edema in the injected joint. Body weight is recorded upon arrival, before study initiation and once a week thereafter according to Pharmaseed's SOP No. 010 “Weighing Laboratory Animals.”
Incapacitance (weight bearing test): weight-bearing changes in rats with monoarthritis are measured using an incapacitance tester, according to Pharmaseed's SOP No. 128 “Use of the Incapacitance Apparatus.” Postural imbalance, which reportedly indicates a change in the pain threshold and weight distribution of the limbs, is decreased. Each rat is placed so that each hind paw is resting on a separate force plate on the incapacitance apparatus, and the weight borne by each hind limb will be measured for five seconds. The ratio of the weight borne by the right to left hind limb is calculated. The mean of three consecutive measurements for each rat is recorded.
Weight bearing function (incapacitance test) is performed on all of the animals at baseline (Day 0) and Days 1, 3, 6 and 10 (total of 5 times per animal).
Tactile Allodynia (Von Frey): rats are placed inside the Plexiglas chamber for a 10-15 minute acclimation period. Subsequently the rats are evaluated for tactile allodynia, according to Pharmaseed's SOP No. 106 “Mechanical Neuropathic Pain Evaluation (Von Frey),” using a Von Frey Filament (VFF) ranging from the thinnest 0.6 g filament up to the thickest 15 g filament (0.6, 1.4, 2, 4, 6, 8, 10, 15 g) in the following manner: the technician approaches the animal from below using the thinnest assorted von Frey filament and touches the hind paw five consecutive times, or until the rat responds. If no response occurs, proceed to the next ascending filament. Once a withdrawal response is established, the paw is retested, with the preceding descending filament until no response is observed. The lag time between filaments, ascending or descending is approximately 90 seconds. Each animal has both hind paws tested in this manner (first the injected leg and then the control leg). The lowest amount of force required to elicit a response is recorded as withdrawal threshold in grams. The Von-Frey test will be performed at baseline (Day 0), and on Days 1, 3, 6 and 10 (total of 5 times per animal).
At termination, animals are sacrificed by CO2 asphyxiation and gross pathology is performed examining the local injection site, major tissue and organ systems according to Pharmaseed SOP No. 007: Necropsy and post mortem examination. Each animal terminating the study as scheduled or terminating the study earlier, either through euthanasia or found dead in their cage, undergoes gross pathology examination. In addition, all gross lesions in organs and major tissue are documented.
Numerical results are given as means and standard deviation or standard error of the mean. The results are subjected to either t-test and/or ANOVA analysis followed by contrast analysis between the groups whenever appropriate, using GraphPad Prism 5 software. A probability of 5% or less (p≤0.05) will be regarded as statistically significant.

### Example 2. Evaluation of the Paracetamol and N-Palmitoylethanolamide Combination in a Murine SNI Model (Neuropathic Pain/Postoperative Pain

The objective of the study is to evaluate the potential of a paracetamol-sparing effect of PEA in a postoperative murine model (SNI model).
Peripheral neuropathic pain is a severe chronic pain condition which may result from trauma to sensory nerves in the peripheral nervous system. The spared nerve injury (SNI) model induces symptoms of neuropathic pain such as mechanical allodynia i.e. pain due to tactile stimuli that do not normally provoke a painful response. The SNI mouse model involves ligation of two of the three branches of the sciatic nerve (the tibial nerve and the common peroneal nerve), while the sural nerve is left intact. The lesion results in marked hypersensitivity in the lateral area of the paw, which is innervated by the spared sural nerve. The non-operated side of the mouse can be used as a control. The advantages of the SNI model are the robustness of the response and that it doesn't require expert microsurgical skills. The threshold for mechanical pain response is determined by testing with von Frey filaments of increasing bending force, which are repetitively pressed against the lateral area of the paw. A positive pain reaction is defined as sudden paw withdrawal, flinching and/or paw licking induced by the filament. A positive response in three out of five repetitive stimuli is defined as the pain threshold.
Animal handling is performed as described in Example 1.
Study Design: mice are randomly allocated to cages on the day of reception. Allocation to relevant groups is done on Day 0, according to pain response testing results. Animals are allocated to seven treatment groups as indicated below:

1M Control (naïve)

2M Contol (induced)

3M Paracetamol (regular dose (RD))

4M Paracetamol (sub-effective (SED))

5M PEA

6M Paracetamol RD with PEA

7M Paracetamol SED with PEA

Mice are placed inside the Plexiglas chamber for a 10-15 minute acclimation period. Subsequently the mice are evaluated for tactile allodynia, according to Pharmaseed's SOP No. 106 “Mechanical Neuropathic Pain Evaluation (Von Frey),” using a Von Frey Filament (VFF) ranging from the thinnest 0.6 g filament up to the thickest 15 g filament (0.6, 1.4, 2, 4, 6, 8, 10, 15 g) in the following manner: the technician approaches the animal from below using the thinnest assorted von Frey filament and touches the hind paw 5 consecutive times, or until the rat responds. If no response occurs, proceed to the next ascending filament. Once a withdrawal response is established, the paw is retested, with the preceding descending filament until no response is observed. The lag time between filaments, ascending or descending is approximately 90 seconds. Each animal has both hind paws tested in this manner (first the injected leg and then the control leg). The lowest amount of force required to elicit a response is recorded as withdrawal threshold in grams. Von-Frey test will be performed at baseline (Day 0), and on Days 1, 3, 6 and 10 (total of 5 times per animal).
At termination, animals are sacrificed by CO2 asphyxiation and gross pathology is performed examining the local injection site, major tissue and organ systems according to Pharmaseed SOP No. 007: Necropsy and post mortem examination. Each animal terminating the study as scheduled or terminating the study earlier, either through euthanasia or found dead in their cage, undergoes gross pathology examination. In addition, all gross lesions in organs and major tissue are documented.
Numerical results are given as means and standard deviation or standard error of the mean. The results are subjected to either t-test and/or ANOVA analysis followed by contrast analysis between the groups whenever appropriate, using GraphPad Prism 5 software. A probability of 5% or less (p<0.05) will be regarded as statistically significant.

### Example 3. Evaluation of the Paracetamol and N-Palmitoylethanolamide Combination in a Rat Fever Model (Induced by Baker's Yeast or 2, 4-Dinitrophenol (DNP))

The objective of the study is to evaluate the potential of a paracetamol-sparing effect of PEA in a model of fever in animals.
Fever is one of important clinical manifestations at the process of many diseases. The high body temperature induces disorder in the internal environment, and accelerates a change in cell responses and metabolism so that metabolic networks change accordingly.
Animal handling is performed as described in Example 1.
Study Design: Two different models were used to induce a fever model, the baker's yeast-induced fever model and DNP model. For the baker's yeast model, the rats are randomly divided into seven groups each consisting of six animals (n=6). Allocation to relevant groups is done on Day 0, according to pain response testing results. Animals are allocated to seven treatment groups as indicated below:

1M Control (naïve)

2M Contol (induced)

3M Paracetamol (regular dose (RD))

4M Paracetamol (sub-effective (SED))

5M PEA

6M Paracetamol RD with PEA

7M Paracetamol SED with PEA

All the groups are first treated with baker's yeast (Saccharomyces cerevisiae) (3 mL/kg of 10% suspension subcutaneous) for fever induction. After 4 hours of yeast administration, animal groups are treated with either vehicle or tested samples. The dose of C. scaposa is selected by an effective dose fixation study method with slight modification. Rectal temperature is measured with digital thermometer coated with glycerin as a lubricant. After baker's yeast injection, rectal temperature is recorded once hourly. The animals which are showing a rise in temperature of 0.5-1° C. during the fourth hour are included in the study. After four hours of yeast injection, all the tested samples are administered orally with the help of a syringe. After medicine administration, rectal temperature is recorded one hourly for 6 hours.
For the 2, 4-dinitrophenol (DNP) model, male Wistar rats (6 weeks age; weight: (200±20) g) are utilized. The rats are randomly divided into seven groups each consisting of six animals (n=6). Allocation to relevant groups is done on Day 0, according to pain response testing results. Animals are allocated to seven treatment groups as indicated below:

1M Control (naïve)

2M Contol (induced)

3M Paracetamol (regular dose (RD))

4M Paracetamol (sub-effective (SED))

5M PEA

6M Paracetamol RD with PEA

7M Paracetamol SED with PEA

The basal oral temperature of rats fasted for 12 hours is recorded. Pyrexia is then induced by intraperitoneal injection of 2,4-DNP (prepared at a concentration of 1 mg/ml in 0.9% sodium chloride solution) at a dose of 20 mg kg-1 (Berkan, 1991). After the confirmation of hyperthermia 30 minutes and after 2,4-DNP administration, treatment is then carried out orally in all tested groups of six animals each as outlined.

1. A pharmaceutical composition comprising a therapeutically-effective amount of a mixture of at least one derivative of 4-Aminophenol or a salt thereof and at least one N-acylethanolamine or a salt thereof.

2. The pharmaceutical composition of claim 1, comprising about 0.5 mg to about 4000 mg of the at least one derivative of 4-Aminophenol or salt thereof.

3. The pharmaceutical composition of claim 1, wherein the at least one derivative of 4-Aminophenol is paracetamol.

4. The pharmaceutical composition of claim 3, wherein the pharmaceutical composition comprises about 10 mg, about 48 mg, about 80 mg, about 120 mg, about 160 mg, about 325 mg, about 500 mg, or about 650 mg of the paracetamol.

5. The pharmaceutical composition of claim 1, wherein the pharmaceutical composition comprises about 800 mg of the N-acylethanolamine or salt thereof.

6. The pharmaceutical composition of claim 1, wherein the at least one N-acylethanolamine is chosen from N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, and any combination thereof.

7. The pharmaceutical composition of claim 6, wherein the at least one N-acylethanolamine is PEA or salt thereof.

8. The pharmaceutical composition of claim 1, wherein the mixture comprises paracetamol or a salt thereof and PEA or a salt thereof.

9. The pharmaceutical composition of claim 8, wherein the mixture comprises about 0.5 mg to about 4000 mg of the paracetamol or salt thereof and about 50 mg to about 5000 mg of the PEA or salt thereof.

10. The pharmaceutical composition of claim 1, wherein the pharmaceutical composition is formulated for systemic administration.

11. The pharmaceutical composition of claim 10, wherein the pharmaceutical composition is formulated for oral, vaginal, rectal, oral mucosal, nasal, sublingual, inhalational, topical, parenteral, intravenous, intramuscular, or subcutaneous administration.

12. (canceled)

13. The pharmaceutical composition of claim 1, wherein the pharmaceutical composition is formulated as a solution or as a suppository.

14. A dosage unit comprising the pharmaceutical composition of claim 1.

15. A method for treating pain or fever in a subject in need thereof comprising administering to the subject the pharmaceutical composition of claim 1.

16. The pharmaceutical composition of claim 15, wherein the pain is chosen from an acute pain, chronic pain, and neuropathic pain.

17. A method for treating at least one side-effect associated with paracetamol consumption comprising administering to the subject the pharmaceutical composition of claim 1.

18-19. (canceled)

20. A method of treating pain or fever comprising:
administering to a subject in need thereof a therapeutically-effective amount of at least one derivative of 4-Aminophenol or salt thereof; and
administering to the subject a therapeutically-effective amount of at least one N-acylethanolamine or a salt thereof.

21. The method of claim 20, wherein the therapeutically-effective amount of the at least one derivative of 4-Aminophenol or salt thereof is from about 0.5 mg to about 4000 mg.

22. The method of claim 20, wherein the at least one derivative of 4-Aminophenol is paracetamol.

23. The method of claim 22, wherein the therapeutically-effective amount of paracetamol is about 10 mg, about 48 mg, about 80 mg, about 120 mg, about 160 mg, about 325 mg, about 500 mg, or about 650 mg.

24. The method of claim 20, wherein the therapeutically-effective amount of the at least one N-acylethanolamine or salt thereof is about 800 mg.

25. The method of claim 20, wherein the at least one N-acylethanolamine is chosen from N-palmitoylethanolamine (PEA), Me-Palmitoylethanolamide (Me-PEA), palmitoylcyclohexamide, palmitoylbutylamide, palmitoylisopropylamide, oleoylethanolamine (OEA), palmitoylisopropylamide (PIA), salts thereof, and any combination thereof.

26. The method of claim 20, wherein the at least one N-acylethanolamine is PEA or salt thereof.

27. The method of claim 20, wherein the derivative of 4-Aminophenol is paracetamol or a salt thereof and the N-acylethanolamine is PEA or a salt thereof.

28. The method of claim 27, wherein the derivative of 4-Aminophenol and N-acylethanolamine are administered as a mixture comprising about 0.5 mg to about 4000 mg of the paracetamol or salt thereof and about 50 mg to about 5000 mg of the PEA or salt thereof.

29. The method of claim 20, wherein the derivative of 4-Aminophenol and N-acylethanolamine are formulated for systemic administration.

30. The method of claim 20, wherein the derivative of 4-Aminophenol and N-acylethanolamine are formulated for oral, vaginal, rectal, oral mucosal, nasal, sublingual, inhalational, topical, parenteral, intravenous, intramuscular, or subcutaneous administration.

31. (canceled)

32. The method of claim 20, wherein the derivative of 4-Aminophenol and N-acylethanolamine are formulated as a solution or as a suppository.

33. The method of claim 20, wherein at least one side-effect associated with paracetamol consumption is prevented or treated.

34. The method of claim 20, wherein the derivative of 4-Aminophenol and N-acylethanolamine are orally administered.

35. The method of claim 20, wherein the derivative of 4-Aminophenol and N-acylethanolamine are administered daily.

36. The method of claim 20, wherein the derivative of 4-Aminophenol and N-acylethanolamine are comprised in the same pharmaceutical composition.

37. A kit for the treatment of pain or fever comprising:
a pharmaceutical composition comprising a therapeutically-effective amount of at least one derivative of 4-Aminophenol or a salt thereof; and
a pharmaceutical composition comprising a therapeutically-effective amount of at least one N-acylethanolamine or a salt thereof.
